(19) United States
(12) Patent Application Publication
(10) Pub. No.: US 2016/0299167 A1
(43) Pub. Date: Oct. 13, 2016

(54) Analyzer

(71) Applicant: HITACHI HIGH-TECHNOLOGIES CORPORATION, Tokyo (JP)

(72) Inventor: Minoru SANO, Tokyo (JP)

(21) Appl. No.: 15/100,399

(22) PCT Filed: Dec. 10, 2014
(86) PCT No.: PCT/JP2014/082605

§ 371 Date: May 31, 2016

(30) Foreign Application Priority Data
Dec. 27, 2013 (JP) 2013-270800

(51) Int. Cl. G01N 35/10; C12Q 1/68

(52) U.S. Cl. CPC G01N 35/1016; C12Q 1/686; G01N 2035/1025; G01N 2035/00435

(57) ABSTRACT

Provided is an analyzer (1) that is provided with a liquid level detection function or a droplet collection function and a cold retention function; efficiently uses a reagent, sample, or other liquid collected in a container (22); and carries out highly accurate analysis. This analyzer (1) is provided with a dispenser (2), a dispensing tip (21) attached to the dispenser (2), a container (22) for containing liquid sucked in by the dispenser (2), a holder (23) for holding the container (22), a conductor (24) covering the outer surface of the container, a control unit (4) for detecting the capacitance between the dispensing tip (21) and the conductor (24) and/or controlling the electric field between a pair of electrodes (41, 42), and a cold retention device (60) for keeping the container (22) cold. The container (22) has at least two cross-sectional shapes (S1, S2) for which the cross-sectional areas in the horizontal direction are smaller at deeper positions.

### TECHNICAL FIELD

The present invention relates to an analyzer having a liquid level detection function for sucking in and ejecting liquid.

### BACKGROUND ART

Conventionally, there have been liquid level detection devices for detecting a liquid level in order to suck in a desired amount of liquid from liquid contained in a container, which detect the liquid level on the basis of a change in capacitance between a pair of electrodes. In order to detect the liquid level, for example, a technology that detects a change in capacitance between a probe for carrying out dispensing and a conductor placed outside the container has been developed.

### CITATION LIST

### Patent Literatures

PTL 1: JP-A-2011-22041

PTL 2: JP-A-8-94642

### SUMMARY OF INVENTION

### Technical Problems

In a liquid level detection device disclosed in PTL 1, electrodes paired with a probe cover at least an outer wall of a bottom portion in an outer wall of a container containing liquid. However, when the liquid is repeatedly sucked in by the probe and an amount of the liquid contained in the container is reduced, a change in capacitance becomes small, and therefore it is difficult to detect a liquid level. As a result, a timing and a depth at which the probe is brought into contact with the liquid level are changed, and therefore a suction amount of the liquid is incorrect.

This invention has been made in view of the above problems, and an object of this invention is to provide an analyzer having a liquid level detection function for sucking in a small amount of liquid contained in a container more accurately.

### Solution to Problems

In order to achieve the above object, an analyzer of the invention includes: a dispensing mechanism for sucking in and ejecting liquid, the dispensing mechanism having a liquid level detection function; a container containing unit; electrodes; and a detection unit for detecting that the dispensing mechanism is in contact with the liquid by measuring a change in capacitance between the dispensing mechanism and the electrodes, in which the container containing unit has two or more different opening shapes in a depth direction.

### Advantageous Effects of Invention

According to the above configuration, it is possible to provide an analyzer having a liquid level detection function for sucking in a small amount of liquid contained in a container more accurately.

### BRIEF DESCRIPTION OF DRAWINGS

FIG. 1 illustrates an analyzer and a dispenser.

FIG. 2 illustrates an analyzer and a dispenser.

FIG. 3 illustrates a cold retention device.

FIG. 4 illustrates a cold retention device.

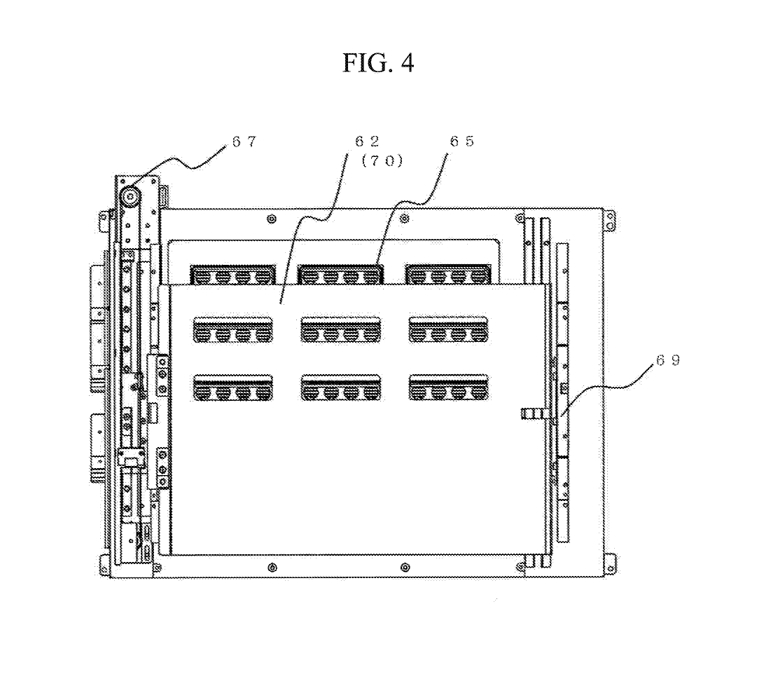

### DESCRIPTION OF EMBODIMENTS

Hereinafter, preferred embodiments of the invention will be described with reference to drawings.

The invention includes a container which contains liquid and whose cross-sectional area in a horizontal direction is gradually changed, a holder for holding the container, electrodes whose internal shapes are gradually changed to be in close contact with the container in accordance with a change in the cross-sectional area of the container, and a dispenser for sucking in and ejecting liquid, the dispenser being a device whose portion to be brought into contact with the liquid is a conductor.

In the invention, the container whose cross-sectional area is gradually changed is such that the cross-sectional area is smaller in the bottom portion.

In the invention, the electrodes which are in contact with the container are changed in accordance with a change in a container shape in a depth direction.

In the invention, the containers and the electrodes for detecting a liquid level are provided in a cold retention device having a cold retention function in order to keep cold samples and liquid medicines stored in the containers.

In the invention, a member for holding the samples and the liquid medicines is divided.

According to the above configuration, when a probe for sucking in liquid approaches the container containing the liquid to be brought into contact with a liquid level, a liquid level detection function can recognize that the probe has been brought into contact with the liquid level, and the probe can suck in the liquid while being sunk at a necessary depth. Because this positional relationship is reproduced for each suction operation, an accurate suction amount is achieved.

When samples and liquid medicines are kept cold, a storage period of the liquid medicines in an analyzer is increased. This makes it possible to efficiently use the samples and the liquid medicines.

Because the member for holding the samples and the liquid medicines is divided into a plurality of parts, in a process in which the analyzer automatically carries out analysis, removal of a container is carried out such that a member holding a group of sample/liquid medicine is fixed while being used for analysis so as not to be pulled out by a user, whereas the member holding the group of the sample/liquid medicine is pulled out by the user when the analysis is completed, and a new container containing a sample/a liquid medicine is placed. With this, it is possible to efficiently carry out analysis processing without inhibiting an automatic operation of the analyzer.

### Example 1

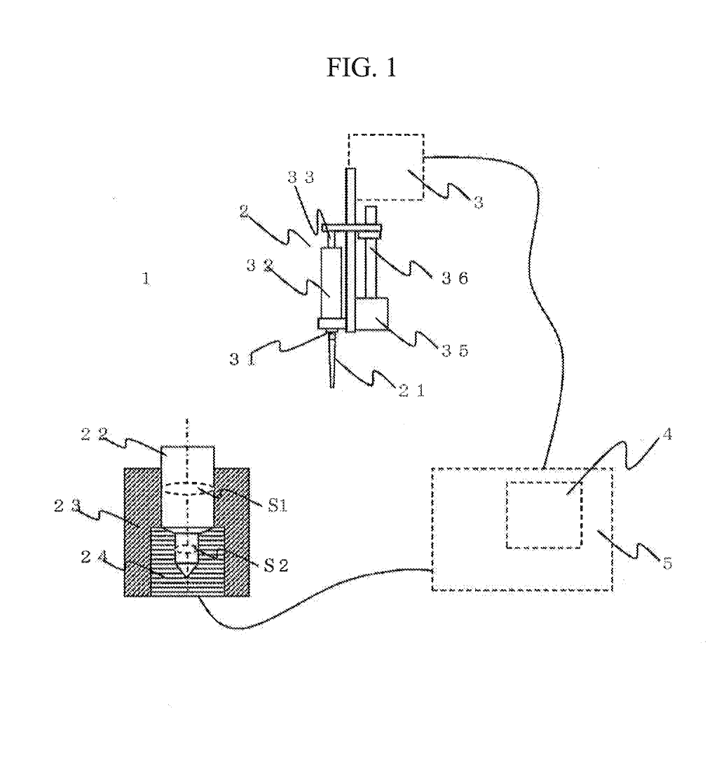

FIG. 1 illustrates an embodiment of an analyzer 1 including a liquid level detection device, which forms the invention. The analyzer 1 includes a dispensing tip 21 attached to a dispenser 2, a container 22 for containing liquid to be sucked in by the dispenser 2, a holder 23 for holding the container, and a conductor 24 covering an outer surface of the container 22.

The dispenser 2 includes a nozzle 31 to which the dispensing tip 21 is attached, a syringe 32 communicating to the nozzle 31, a plunger 33 connected to the syringe 32, and a moving mechanism 34 for moving the plunger 33. As the moving mechanism 34 of the plunger 33, for example, a combination of a stepping motor 35 and a ball screw 36 can be appropriately selected. Although air is used as a pressure medium for sucking in and ejecting liquid in this embodiment, it is possible to use water as a pressure medium by filling a communicating flow channel with water. A robot arm 3 that can be moved in a space is provided, and the dispenser 2 fixed to the robot arm 3 can be freely moved in the analyzer 1.

The dispenser 2 is electrically connected from a tip end of the chip to a control unit 4 of the analyzer 1 to which the dispenser 2 is attached, and, similarly, the conductor 24 covering the outer surface of the container 22 is electrically connected to the control unit 4 of the analyzer 1.

An operation in which the analyzer 1 sucks in liquid will be described. The dispenser 2 is horizontally moved by the robot arm 3 to a position where target liquid can be sucked in. Then, the dispenser 2 falls in a vertical direction, and, before the tip end of the dispensing tip 21 is brought into contact with a liquid level, the dispensing tip 21 and the conductor 24 covering the outer surface of the container 22 act as facing electrodes, and capacitance C1 is detected in the control unit 4 and is then stored in a storage 5. The dispenser 2 further falls, and, when the dispensing tip 21 and a liquid surface are brought into contact with each other, the liquid is electrically connected to the dispensing tip 21. Therefore, the capacitance C1 between the liquid and the dispensing tip 21 and the conductor 24 is increased, as compared with C0, and C1 is detected in the control unit 4 and is compared with C0. Thus, the analyzer 1 recognizes that the dispenser 2 has been brought into contact with the liquid level. A form of a calculation method in the control unit 4 for detecting a liquid level on the basis of a change in capacitance is disclosed in, for example, JP-A-8-94642.

Herein, the dispensing tip 21 is made of plastic having electrical conductivity because, for example, the plastic contains carbon and is exchanged in the interval between suction of liquid and ejection thereof so as not to contaminate liquid to be sucked in. Alternatively, the dispenser 2 may include a nozzle 31 that does not use the dispensing tip 21, is made of an electrically conductive material that is electrically connected, and is not exchanged.

Suction of liquid is started at a position where the tip end of the dispensing tip 21 of the dispenser 2 is sunk from a liquid level at a certain depth. An amount of fall of the dispenser is controlled so that the tip end of the dispensing tip 21 can still stay in the liquid contained in the container 22 after a desired amount of the liquid is sucked in. Because the tip end of the dispensing tip 21 is in the liquid while the dispenser 2 is sucking in the liquid, it is possible to prevent suction of air and carry out accurate suction and ejection (hereinafter, referred to as “dispensing”).

The container 22 containing liquid is stably held by the holder 23. The conductor 24 used in the liquid level detection device is fixed to the holder 23 and is positioned to be in contact with the outer wall of the container 22. The conductor 24 is preferably, for example, a cushioning conductive gasket and is brought into close contact with the outer wall of the container 22 at a position where the container 22 is fixed. As compared with the case where the conductor and the container are placed with a gap, a distance between the facing electrodes is reduced. This increases a change in capacitance before and after the dispensing tip 21 is brought into contact with the liquid level, and therefore it is possible to improve detection accuracy. Further, when a fastener for holding the container 22 is provided to bring the container 22 into contact with the conductive gasket, this effect can be secured. In order to securely bring the conductor 24, the fastener, and the like into close contact with the container 22, an elastic body such as a spring can be provided.

The container 22 has a shape whose cross-sectional area is gradually changed in a depth direction. The container 22 has a certain cross-sectional shape S1 from an opening portion to a certain depth, a region whose cross-sectional area in the horizontal direction is changed to connect different cross-sectional shapes, a certain cross-sectional shape S2 at a position deeper than this region (at a downward position), and a V-shaped bottom portion. Herein, an area of the cross-sectional shape S1 is larger than that of the cross-sectional shape S2.

When an amount of liquid contained in the container 22 is sufficient, the tip end of the dispensing tip 21 can be positioned at a certain depth below the liquid level before and after the liquid is sucked in. When dispensing is repeatedly carried out and the amount of the liquid is reduced, if the container has only the cross-sectional shape S1, a bottom surface and the dispensing chip are brought into contact with each other and a sufficient depth cannot be obtained. However, as in this example, the container has a region of the cross-sectional shape S2 whose cross-sectional area is smaller than that of the cross-sectional shape S1, and therefore the dispensing chip can secure a certain depth with respect to the liquid level when the liquid is sucked in. Further, because the bottom portion has a V shape, a gap for sucking in the liquid is secured between the bottom portion and the tip end of the dispensing tip 21 even in the case where the tip end of the dispensing chip is in contact with the container 22. Therefore, the dispensing tip 21 still stays at a certain depth from the liquid level when suction of the liquid is completed. Thus, it is possible to not only accurately reproduce a suction amount but also reduce the liquid remaining in the container 22 and efficiently use the liquid.

At this time, because the tip end of the dispensing tip 21 is below the liquid level while the liquid is being sucked in, it is possible to have a resistance to movement of the liquid level caused by vibration of the analyzer. This makes it possible to prevent reduction in accuracy caused by suction of air.

The shape of the container is not limited to a combination of two cross-sectional shapes and can be any of various combinations. That is, an optimal combination can be employed in accordance with a method of packaging the container, a shape of the holder, and the like.

Positions of the dispensing chip and the container in the horizontal direction have errors such as a drive error of the robot arm, deflection of the dispenser, and mounting errors of the dispensing chip and the nozzle. In the case where the tip end of the dispensing chip cannot stop at an accurate position in the dispensing container, a positional relationship between the dispensing chip and the conductor is changed, which results in an error in detection of capacitance. Thus, the container is caused to have a shape for correcting the position of the dispensing chip, and therefore the error can be reduced.

Capacitance can be increased as a surface area is increased. Therefore, when the conductor, which can be brought into contact with the outer wall of the container, is placed to be brought into contact with an outer surface shape of the container with no gap, a resistance to a noise or the like can be enhanced.

As compared with a state in which the amount of the liquid is sufficient, in a state in which a residual amount of the liquid is small (for example, a state in which the liquid level positions in the region having the cross-sectional shape S2), accuracy of liquid level detection is further demanded to accurately suck in the liquid. Because the amount of the liquid is small, surface areas of the dispensing chip and the liquid, which serve as electrodes, are reduced, and a change amount of capacitance is also reduced accordingly. Therefore, the conductor is effectively provided to cover at least a shape of and around the bottom portion of the container.

Further, in order to increase a change in the capacitance, it is possible to reduce a thickness of the whole container or a thickness of a part in the region of and around the bottom portion of the container, the region being a region where accuracy is particularly demanded. Further, the whole container or the part in the region of and around the bottom portion of the container can be made of a material having high permittivity.

A container acting as a conductor, which is obtained by causing an electrically conductive material to adhere to an outer surface shape of the container, may be used. It is possible to form an electrode by providing a member for electrically connecting the conductor to the holder and installing the container in the holder. Herein, the member is made of a spring steel to securely bring the conductor portion of the container into contact with the holder, and therefore conduction can be achieved.

### Example 2

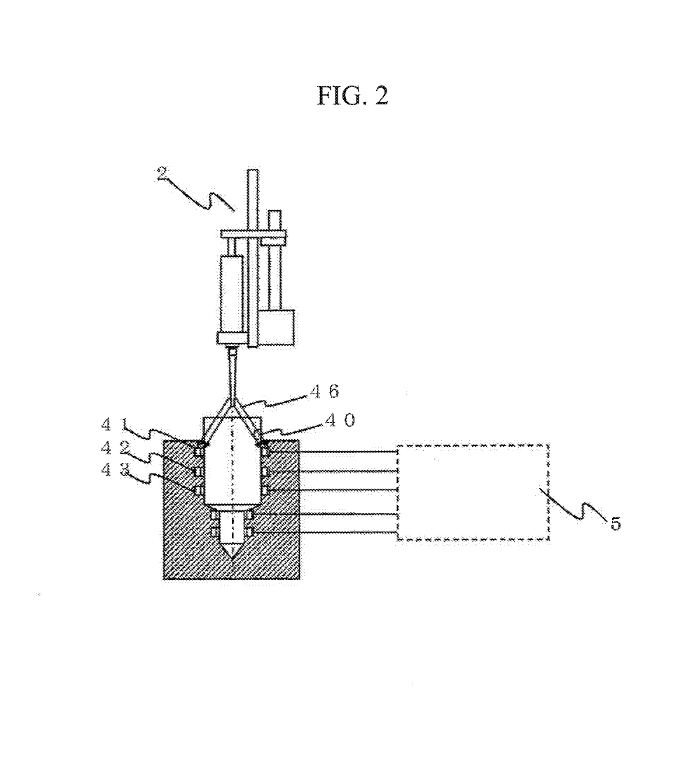

In the configuration in Example 1, a conductor having a structure that covers the bottom portion of the container is provided instead of the conductor covering the outer shape of the container, and therefore a new effect is exerted. This will be described with reference to FIG. 2. The dispensing chip of the dispenser falls in the container, and a voltage is applied so that the dispensing chip has a positive charge and the conductor has a negative charge, thereby generating an electric field. A combination of positive and negative charges may be reversed, or a negative charge can be grounded. In the case where liquid is an aqueous solution, the solution exposed to the electric field is captured by electrostatic force. Herein, it is assumed that the liquid remains on the inside of the container in the form of droplets. For example, when the solution contained in the container is sucked in/ejected, a droplet 40 adheres to an inner wall surface and remains thereon in some cases. Further, when a user carries the container in order to place the container in the analyzer or when a user places the container in the analyzer, the liquid contained in the container is not collected in the bottom of the container but remains on the inner wall surface due to vibration, impact, or the like in some cases. As a shape of the container for opening the container, for example, there is considered a configuration in which a lid is provided and the lid is removed when the container is installed in the analyzer. In addition, the following configurations are considered as alternatives: a configuration in which an opening portion is sealed by a film such as a plastic film or an aluminum film and, when the container is installed in the analyzer, the film is removed or is perforated with a drill to make a hole for allowing the dispensing chip to pass therethrough; and a configuration in which a slit film made of a plastic material such as rubber is provided and allows the dispensing chip to pass therethrough.

The solution is captured by the dispensing chip and the conductor, and the dispensing chip further falls. The electric field formed by the dispensing chip and the conductor falls as the dispensing chip falls, thereby bringing about an effect that drops the solution downward. With this, the solution that has adhered to the wall surface of the container is collected in the bottom of the container. This reduces the solution that cannot be sucked in and remains in the container. That is, the liquid can be efficiently used.

The electrostatic force for capturing the solution is the strongest when a distance between the dispensing chip and the conductor is the shortest. Therefore, a position and a size of the conductor are set so that the solution to be collected in the bottom of the container is captured between the dispensing chip and the conductor at a position where a distance between those electrodes is reduced as much as possible. Therefore, for example, as described in the head of the description, it is preferable to place, in the container having two cross-sectional shapes, a conductor that is brought into contact with an outer surface shape of a smaller portion around the bottom portion.

With this, the solution is collected in the bottom portion, and therefore highly accurate suction can be achieved.

As a configuration for forming electrodes and collecting a solution adhering to the wall surface in the bottom of the container, the following structure is also considered. A plurality of arrays of electrodes are formed on the outer surface shape of the container. The electrodes are electrically independent from each other from an upper side toward a lower side of the container and are connected to the control unit of the analyzer. A desired voltage or grounding can be applied to each of the electrodes by the control unit.

A positive charge is applied to an upper electrode 41, and an electrode 42 positioning therebelow is grounded, and therefore an electric field 46 (lines of electric force are illustrated) is generated between the electrodes. The solution on the wall surface of the container is captured by the generated electric field. Then, application of positive charges and grounding to the electrodes forming the electric field generated first are stopped, and an electrically neutral state is formed. At the same time, a positive charge is applied to the electrode 42 that is below the electrode 41 to which a positive charge has been applied first and has been grounded first, and an electrode 43 therebelow is grounded. The generated electric field is below the electric field generated first, and electrostatic force for attracting the solution that has been captured by the electric field generated first is generated. Therefore, an effect that drops the solution downward is exerted. As described above, a pair of electrodes for generating an electric field is changed downward, and therefore the solution adhering to the outer wall of the container is collected in the bottom portion of the container. This contributes to reduce waste of the solution.

Herein, as another means for collecting the liquid in the bottom portion of the container, it is possible to select a mechanism for applying vibration or centrifugal force to the container.

### Example 3

There will be described an embodiment in which a structure for keeping cold liquid contained in the container is added to the analyzer in Example 1 or Example 2. The analyzer uses, for example, a polymerase chain reaction (hereinafter, referred to as “PCR”) method as a technique for quantitating a target gene contained in a sample. In the PCR method, it is possible to selectively amplify a desired base sequence by controlling a temperature of a reaction liquid obtained by mixing a sample and a reagent in accordance with a condition determined in advance. An amplification enzyme contained in a reagent to be used for amplifying genes is desirably kept cold in order to prevent deterioration of a property thereof. A temperature for keeping cold the amplification enzyme is, for example, from 2° C. to 8° C.

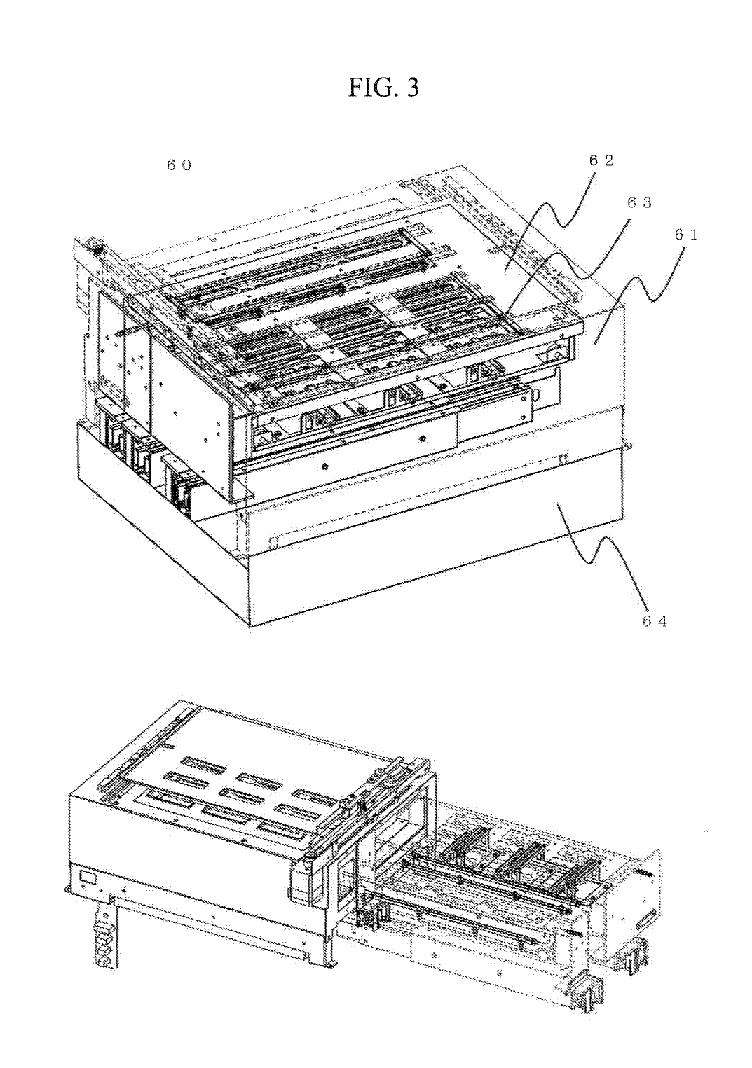

A cold retention device 60 will be described with reference to FIG. 3. The cold retention device includes a housing 61 for keeping cold air inside, a lid 62 for sealing inside, a drawer including a holder 63 for holding the container, and a cooler 64 for blowing cold air to the cold retention device. Parts of the housing, the lid, and the cooler, the parts being in contact with open air, include a thermal-insulation material in order to improve a thermal insulation effect. A conductor is provided in the drawer so as to be brought into contact with the outer surface shape of the container fixed by the holder. The conductor is considered to be, for example, a cushioning conductive gasket. Herein, the drawer and the cold retention device are made of metal members, electrically conductive plastics, or the like, and the cushioning conductive gasket is provided in the drawer or the cold retention device so as to be inserted between the drawer and the cold retention device. With this structure, the conductor is electrically grounded in a state in which at least the drawer is received in the cold retention device. The drawer includes a photointerrupter and a dog used for detecting that the drawer is received in the cold retention device, an LED lamp for causing a user to recognize that the drawer is received, and a lock mechanism for preventing the drawer from being pulled out after the drawer is received. The cooler includes a cooling fin for cooling air inside the cold retention device, a Peltier element fixed so that a cooling surface is brought into contact with the cooling fin, a radiating fin for radiating heat, the radiating fin being placed on a thermal-radiation surface of the Peltier element, a motor fan for radiating heat of the radiating fin, a motor fan for circulating cooled air inside the cold retention device through the cold retention device, and a drain for discharging condensation generated on the cooling fin. A space in the cold retention device for receiving the container and a space for cooling air of the cooler communicate to each other, and cooling air cooled in the cooler is blown to the space in the cold retention device for receiving the container, and air warmed in the space in the cold retention device returns to the cooler and is then cooled. The cold retention device includes a temperature sensor, and the control unit controls output of the cooler on the basis of an output value of the temperature sensor. Therefore, the inside of the cold retention device is kept at a desired temperature.

The cold retention device includes an opening portion 65 through which the dispensing chip of the dispenser dispenses liquid from the received container, a lid, and a mechanism 67 for sliding the lid. Regarding the opening portion, the lid is at an appropriate position (hereinafter, home position) to seal the opening portion in order to keep the liquid cold and reduce evaporation and drying of the liquid while the liquid contained therein is not being dispensed. Meanwhile, in the case where liquid that is set to be used for processing a predetermined analysis item with the use of the analyzer is dispensed, the lid is slid to open a corresponding opening portion, and the analyzer falls and sucks in the desired liquid. Then, the analyzer rises and processes the liquid in accordance with the next step. The lid includes a packing for sealing the cold retention device in a part to be brought into contact with the housing of the cold retention device and a guide 68 including a bearing for sliding the lid. The guide has a wedge shape for generating a downward stroke in the vertical direction when the lid is at the home position and includes a plate spring 69 for pressing the lid downward. When the lid is moved to the home position, the packing is crushed between the lid and the cold retention device to have an appropriate thickness, and therefore a sealing effect is improved. An upper surface of the opening portion of the cold retention device has a projected shape to rim a circumference of the opening portion, and therefore a contact area and repulsive force between the upper surface and the packing of the lid are reduced and a crushing amount of the packing is increased. With this, the sealing effect is improved.

A felt sheet 70 for evaporating condensation water is attached to upper surfaces of the lid and the cold retention device, thereby preventing entry of condensation water through the opening portion.

Herein, a minute gap is provided between the lid and the cold retention device, and a suction port is provided in the cooler. With this, it is possible to have a structure in which cold air of the cold retention device is blown through the gap between the lid and the cold retention device. This makes it possible to prevent generation of condensation in the vicinity of the opening portion.

The drawer includes the holder so as to receive a reagent or a sample determined in advance. A plurality of drawers can be provided, and, for example, containers containing samples can be placed in drawers A and B, and containers containing reagents can be placed in drawers C and D. In order to implement analysis desired by a user, the analyzer automatically operates the dispenser to generate a mixed liquid of a sample placed in the drawer A and a reagent placed in the drawer C and carries out analysis. In the case where a request for analysis is newly made while the analyzer is processing this analysis, it is possible to place a new sample in the drawer B, place a new reagent in the drawer D, and therefore request predetermined analysis via the control unit. Analysis is carried out by the analyzer so that steps, such as generation of a mixed liquid, plugging using a plugging device, mixing using a mixing device, and analysis, are processed in order predetermined in advance. In the case of, for example, the PCR method, the analysis corresponds to steps of adjusting a temperature of a mixed liquid and detecting a change in fluorescence intensity. According to this configuration, it is possible to start processing of analysis that is newly requested while analysis is being processed. Therefore, it is possible to efficiently use units that implement respective processing steps in the analyzer and improve analysis efficiency of the analyzer.

As described above, the analyzer has the cold retention function, and therefore it is possible to improve an analysis property and use a reagent installed once in the analyzer for a longer time. This makes it possible to carry out analysis efficiently.

Herein, regarding the number of drawers and a configuration thereof and a combination of a sample and a reagent which can be installed, an optimal combination can be selected in accordance with an object of the device.

### REFERENCE SIGNS LIST

1 . . . analyzer, 2 . . . dispenser, 3 . . . robot arm, 4 . . . control unit, 5 . . . storage, 21 . . . dispensing chip, 22 . . . container, 23 . . . holder, 31 . . . nozzle, 32 . . . syringe, 33 . . . plunger, 34 . . . plunger moving mechanism, 35 . . . stepping motor, 36 . . . ball screw, 41 . . . electrode, 42 . . . electrode, 43 . . . electrode, 46 . . . electric field, 60 . . . cold retention device, 61 . . . housing, 62 . . . lid, 63 . . . holder, 64 . . . cooler, 65 . . . opening portion, 67 . . . opening and closing mechanism of lid, 68 . . . guide, 69 . . . plate spring, 70 . . . felt sheet

1. An analyzer, comprising:
a dispensing mechanism for sucking in and ejecting liquid, the dispensing mechanism having a liquid level detection function;
a container containing unit;
electrodes; and
a detection unit for detecting that the dispensing mechanism is in contact with the liquid by measuring a change in capacitance between the dispensing mechanism and the electrodes, wherein
the container containing unit has two or more different opening shapes in a depth direction.

2. The analyzer according to claim 1, wherein
the electrodes are provided in a bottom portion of the container containing unit.

3. The analyzer according to claim 2, wherein
among the opening shapes in the depth direction, the electrodes are provided to cover the opening shape positioned in a bottommost portion.

4. The analyzer according to claim 1, wherein
the electrodes are provided in the container containing unit so as to be electrically independent from each other in the depth direction.

5. The analyzer according to claim 4, comprising
a control unit for controlling an electric field of the electrodes, wherein
a pair of electrodes for generating the electric field is changed from an upper side toward a lower side.

6. The analyzer according to claim 1, comprising
a cold retention device receiving a container.

7. The analyzer according to claim 1, wherein
each electrode is a cushioning conductive gasket.

8. The analyzer according to claim 1, wherein
a thickness of and around a bottom portion of a container is smaller than a thickness of the other parts.

9. The analyzer according to claim 1, wherein
permittivity of a material of and around a bottom portion of a container is higher than permittivity of a material of the other parts.
